(19) United States
(12) Patent Application Publication
(10) Pub. No.: US 2003/0175248 A1
(43) Pub. Date: Sep. 18, 2003

(54) Regenerative adjuvant

(76) Inventor: Gunter Uhr, Seerosenstrasse (DE)

Correspondence Address: ROSENTHAL & OSHA L.L.P., 1221 MCKINNEY AVENUE, SUITE 2800, HOUSTON, TX, 77010, US

(21) Appl. No.: 10/149,989

(22) PCT Filed: Dec. 15, 2000
(86) PCT No.: PCT/EP00/12803

(30) Foreign Application Priority Data
Dec. 15, 1999 (DE) 199 60 504.1

(51) Int. Cl. A61K 45/00; A61K 33/42

(52) U.S. Cl. 424/93.7; 424/602

(57) ABSTRACT

The invention relates to a regenerative adjuvant to be used in skin and bone defects of living beings and to a method that can be carried out in any doctor's practice without the need for a specialized laboratory. The invention further relates to a kit used with this regenerative adjuvant that can be easily handled. The inventive regenerative adjuvant for skin and bone defects comprises thrombocyte-rich plasma (platelet-rich plasma) and calcium phosphates. An inventive method comprises the following steps: drawing up blood into a container, fixing said container in a centrifuge, carrying out the first centrifugation step, removing the container from the centrifuge and the radially inner fraction from the container and transferring it to a second container, fixing said container in the centrifuge, carrying out the second centrifugation step at a substantially higher speed, removing the container from the centrifuge, removing a part of the radially inner fraction from the second container, mixing the radially outer fraction with the rest of the radially inner fraction in the second tube to give PRP plasma and mixing the content of the second container with a bone replacement agent.

### I. FIELD OF USE

The invention relates to a regeneration adjuvant in relation to skin and bone defects on living beings and a method and a kit for the production thereof.

### II. TECHNICAL BACKGROUND

Regeneration adjuvants of that kind are also applied in relation to bone fractures, insertion of dental implants and the like and considerably accelerate the wound and defect healing process. That reduces the stresses from the point of view of the patient, for example by virtue of the fact that temporarily applied metal bars or metal pins can be removed earlier, the necessary duration of walking aids can be reduced and so forth.

### III. STATEMENT OF THE INVENTION

a) Technical Object

Therefore the object of the invention is to provide a regeneration adjuvant of that kind which is particularly simple to produce, in particular in any normal doctor's practice. In addition the manufacturing method is to be such that it can be carried out in any normal doctor's practice without a special laboratory, and the kit to be used for that purpose is also to be designed in such a way that it is correspondingly simple to handle.

Regeneration adjuvants of that kind comprise inter alia thrombocyte-rich plasma, so-called platelet-rich-plasma (PRP). The thrombocytes which are contained in a large number in such PRP liberate various growth factors which control in dependence on dosage the proliferation and activity of target cells such as for example fibrocytes, macrophages, osteoblasts and osteoclasts. In that respect the growth factors can have both a stimulating and also an inhibiting function. A further constituent of regeneration adjuvants of that kind is a synthetically produced substitute or construction material, generally present in powder or granule form or block material. In accordance with the invention this a calcium phosphate, in particular a calcium composite, for example a β-tri-calcium phosphate. A bone construction material of that kind is on the market under the trade name “Cerasorb®”.

In this case the regeneration adjuvant is produced by mixing the bought substitute in granule or powder form with the liquid PRP until the desired consistency, generally permitting plastic shapability, is reached.

In this respect the PRP is preferably to be produced from blood of the patient to be treated with the regeneration adjuvant, from the patient's own freshly removed blood.

The manufacturing method in that respect is such that the blood is firstly centrifuged with a centrifugal force which separates the crude plasma including the thrombocytes contained therein in liquid form as a radially inner fraction from larger solids such as for example erythrocytes and leucocytes which form the radially outward fraction in that first centrifuging step.

The radially inner crude plasma is again centrifuged—generally after being transferred into a second container—, but this time with a markedly higher level of centrifugal force, in particular at least twice the centrifugal force as that used in the first centrifuging step, in particular at least 1300 g, in particular at least 1800 g. As a result the crude plasma is separated almost completely into particle-free liquid plasma as the radially inner fraction and an enrichment of for example thrombocytes and the further solids contained in the crude plasma, as the radially outer fraction, which are scarcely any longer liquid but almost form a solid cake.

To produce the PRP plasma a part and in particular the most part and in particular between about 60 and 80% of the particle-free plasma, that is to say the radially inner fraction, is removed and the remaining residue of the particle-free plasma is shaken up again with the solid cake in the same container to form a thrombocyte-rich plasma (PRP) which is still liquid.

That PRP is then added in a mixing vessel to the substitute or construction material in powder or granule form, for example “Cerasorb®” until the desired consistency is reached.

The above-described manner of manufacturing PRP from blood is known in principle and is executed for example in that manner in large laboratories.

By refining the method and using the correct kit components however it is made possible for that method to be carried out not only in a large laboratory which is set up for same but in any doctor's practice and thus directly in the treatment of skin, bone or tooth defects directly in situ firstly to produce PRP from the patient's own blood taken from the patient, and thus also to manufacture and immediately use the desired regeneration adjuvant. That is achieved in that for example one and the same centrifuge is used for the two centrifuging steps, namely a centrifuge which is present in any doctor's practice, for clamping monovettes or other tubes. In addition for example a monovette is used directly as the first container, the monovette being used to take the blood from the patient, and in particular, by virtue of a desired-rupture location in the plunger rod and the adaptor which is removable from the sterile closure, being suitable for being fitted directly into a suitable centrifuge.

A monovette, in particular with those properties, is equally also used as the second container into which the crude plasma is drawn off from the first container, insofar as firstly a cannula is fitted onto the monovette by means of an adaptor, for piercing the sterile closure of the first container, which is then removed—for centrifuging in the second centrifuging step—, just as the plunger rod is also broken off at the desired-rupture location.

This procedure involves using two identical containers in the form of a monovette, which can thus also both be placed, but in succession, in the same centrifuge which as usual is adjustable in its speed of rotation in order to produce the differing centrifugal forces as are required for the first and second centrifuging steps.

A syringe, a monovette equipped with an adaptor and a cannula or another one of the commercially available tubes used in the medical sector is used for drawing off a part of the particle-free plasma from the second container, as well as for removing the shaken-up PRP.

If in addition in the operation of removing crude plasma or particle-free plasma after the first and the second centrifuging step respectively the volume removed is compensated by the introduction of sterile gas, in particular sterile ambient air, the amount of force involved in drawing off that partial volume is very much less as the plunger position can remain unchanged in the initial container. In particular a cannula with a sterile closure arranged at the free end is used for that purpose. That air cannula with a sterile closure arranged at the free end is inserted in addition to the removal cannula through the sterile closure of the initial container before the operation of removing the partial volumes from the initial container is effected.

Mixing the thrombocytes of which the majority are solid and the remaining particle-free plasma is effected directly in the monovette which is used for the second centrifuging step. In order to permit the liquid PRP which is then ready to be more easily discharged the PRP is preferably not dribbled out of that second monovette into the mixing container by means of the fitted adaptor and the cannula as in that case the retaining force of the sterile closure has to be overcome. Instead it is preferably removed from that second monovette or the container in which the second centrifuging step is carried out with a removal tube which admittedly has a cannula but not a sterile closure of that kind, so that the operation of introducing the PRP of which the majority is liquid into the mixing container in the desired amount can be effected from that removal tube with the application of a small amount of force and thus in a very closely quantitatively controlled fashion.

b) Attainment of the Object

That object is attained by the characterizing features of claim 1. Advantageous configurations are set forth in the appendant claims.

c) Specific Embodiments

An embodiment in accordance with the invention is described in greater detail hereinafter by way of example with reference to the drawings in which:

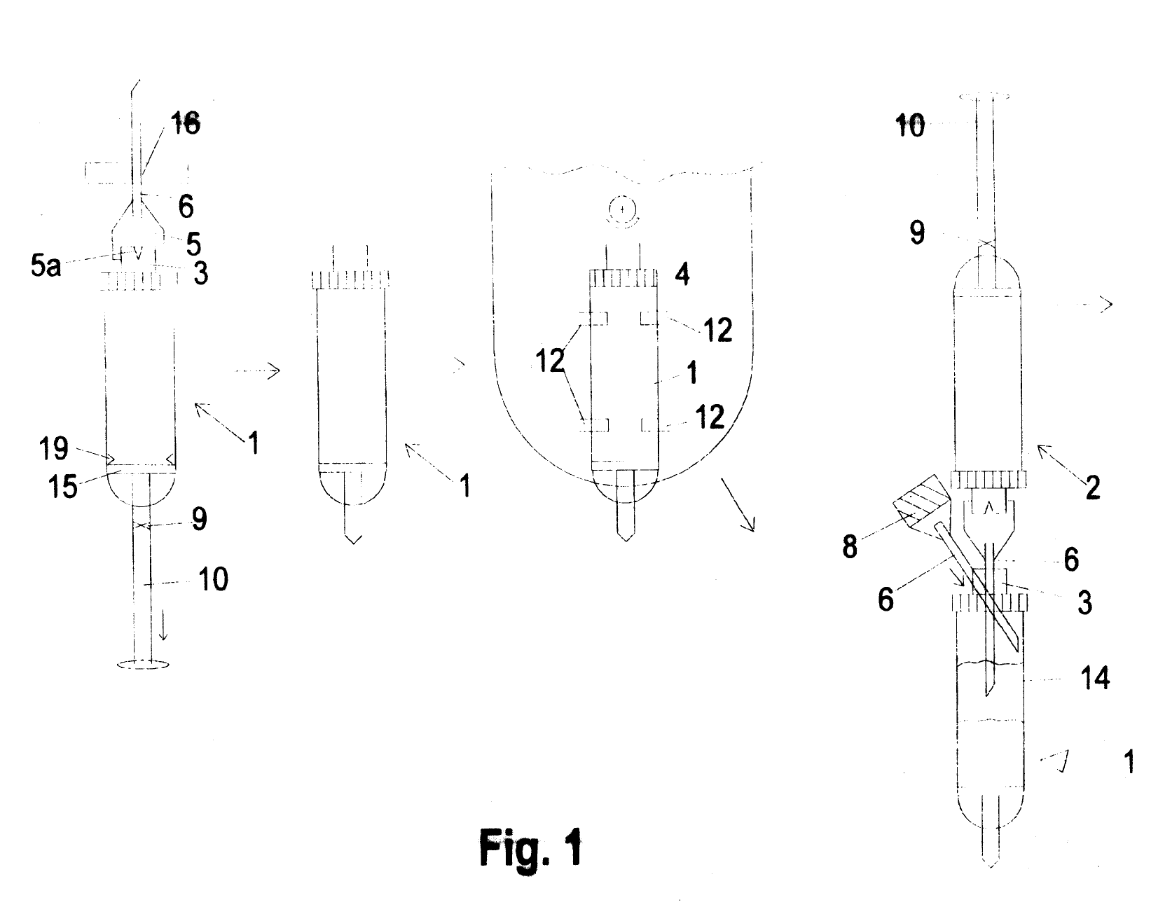

FIG. 1 shows the first method steps, and

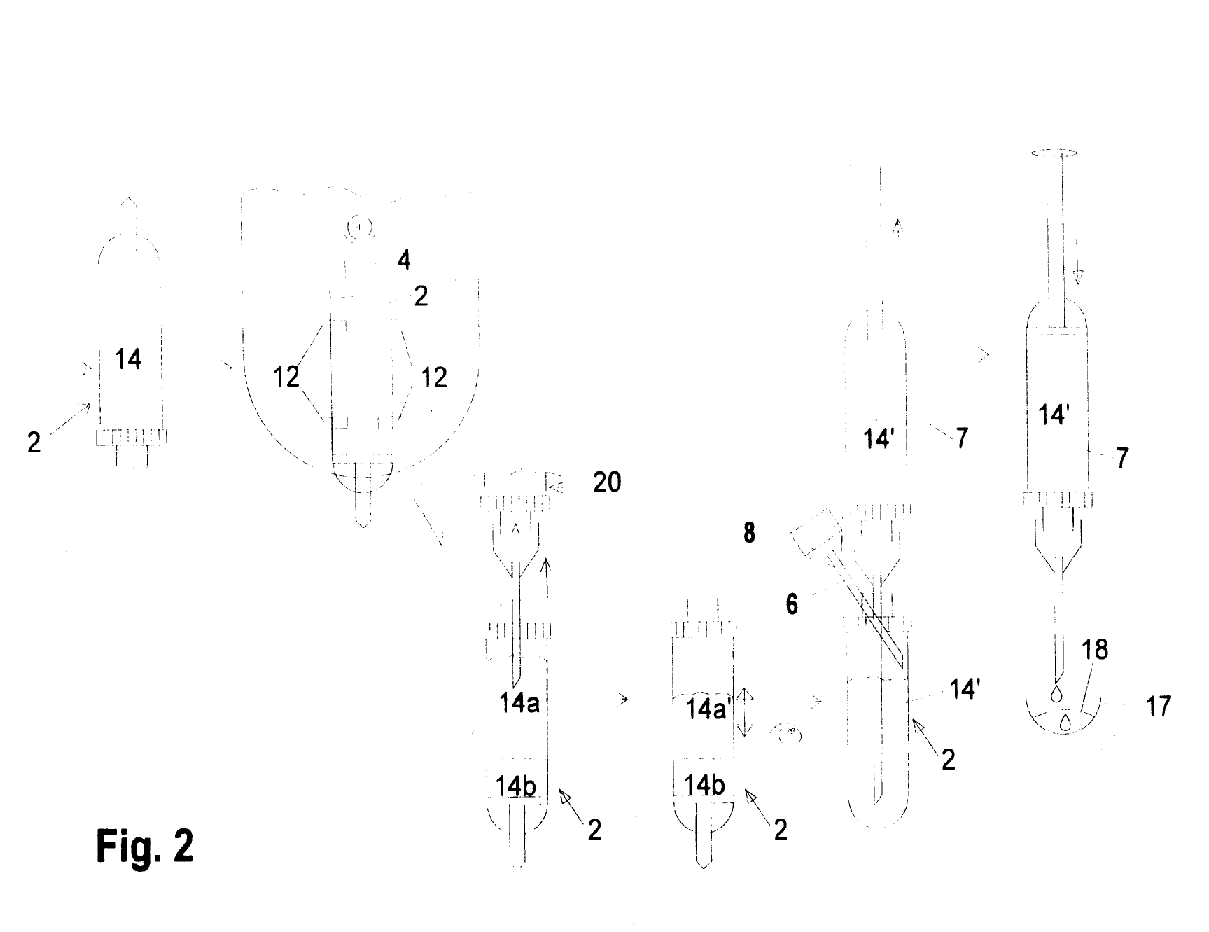

FIG. 2 is a view illustrating the second method steps in principle.

The left-hand side of FIG. 1 shows the first monovette 1 which has a sterile closure 3 which however is pierced by the bar or spike 5a, which acts on the sterile closure from the exterior, of a fitted adaptor 5, and is thus opened. The adaptor 5 in turn carries the cannula 6 which for example is suitable for taking blood from a patient, and in particular is equipped as a so-called butterfly cannula 16 with a transversely extending adhesive strip for remaining on the patient.

In the course of its plunger rod 10 that first monovette 1 has a desired-rupture location 9 afforded by a reduction, at a longitudinal position such that the desired-rupture location 9 is just outside the container part of the monovette 1 when the plunger has arrived at the completely retracted position. In addition, it is recommended that the monovette 1 is so designed that the plunger 15 can be latched in its completely retracted position in a latching means 19, for example in the form of inwardly extending retaining noses 19 which project into the path of movement of the plunger 15. In that way it is possible, after filling of the first monovette 1, to remove therefrom the adaptor 5 and therewith also the cannula 6 or 16 respectively, and on the other hand to break off and remove the outwardly projecting remaining part of the plunger rod 10, at the desired-rupture location 9. The monovette 1 which is reduced in that way is also closed in a sealed and sterile condition at its front end by the sterile closure 3 as same is no longer penetrated by the spike 5a of the adaptor 5.

The preferably completely filled monovette 1 can thus be fitted in the radial direction into a centrifuge 4 and fixed there by means of suitable holders 12, with the sterile closure 3 facing radially inwardly.

After carrying out the first centrifuging step with a low centrifugal force in which the crude plasma including its thrombocytes is separated from the heavier solids of the blood, for example erythrocytes, that radially inner fraction, the crude plasma 14, is removed, as completely as possible, from the first monovette 1 by means of a second monovette 2, the latter again equipped with an adaptor 5 and a cannula 6. In this case the cannula 6 of the second monovette 2 again pierces the sterile closure 3 of the first monovette 1. The cannula 6 is inserted to such an extent that its free end is positioned near the phase limit, but still within the radially inner fraction 14.

In order not to also have to move the plunger 15 of the first monovette 1 when drawing off that crude plasma 14 because of the loss of volume, a further cannula 6 is preferably additionally inserted through the sterile closure 3 of the first monovette 1 at the same time, with an air sterile filter 8 being arranged at the rear, otherwise free end of the further cannula 6 so that after passing the air sterile filter 8 ambient air can subsequently flow into the first monovette 1, as the crude plasma 14 is removed.

The crude plasma 14 which is now in the second monovette 2—as shown in FIG. 2—is—as the next step—fixed in the same centrifuge 4 as the second monovette 2 is preferably identical to the first monovette 1 in its dimensions, at least in terms of fixing it in the centrifuge 4—again with the sterile closure 3 facing radially inwardly, and is there centrifuged in a second centrifuging step. The centrifugal force which now obtains however is markedly higher and in particular is twice as high as in the first centrifuging step so that within the crude plasma the particle-free and thus lighter plasma 14a is positioned radially inwardly with respect to the thrombocytes 14b which contain the heavy solids and which almost form a solid cake.

The most part of the now radially inwardly disposed fraction of the particle-free plasma 14a is drawn off from that second monovette 2 after the second centrifuging step by means of a further tube 20, preferably also again a syringe or monovette, and disposed of, once again with pressure equalization by means of a further cannula 6 with an air sterile filter 8, as described hereinbefore.

In the second monovette 2 the remaining part 14a′ of the particle-free plasma is mixed again by shaking with the concentrated solids of the plasma, in particular the thrombocytes 14b, so that once again the result is a liquid, the PRP, which is highly enriched with thrombocytes and ideally saturated.

That is then added to the substitute or construction agent 18, a granule or powder material, in a mixing vessel 17, until the mixture is of the desired pasty or plastic consistency. As this has to be effected in a very finely quantitatively metered fashion, the PRP is not introduced into the mixing container 17 directly from the second monovette 2, but is firstly removed from the second monovette 2 by means of a removal tube 7 which includes a cannula, in which case once again air is permitted to flow in by way of an air cannula with an air sterile filter at the rear end. The removal tube 7 in this case does not have a sterile closure 3 so that, upon expulsion of the PRP 14′ into the mixing container 17, there is no need to overcome a relatively high force and thus the procedure can be effected in a very finely quantitatively controlled fashion.

List of References

1 first monovette

2 second monovette

3 sterile closure

4 centrifuge

5 adaptor

5a spike

6 cannula

7 removal tube

8 air sterile filter

9 desired-rupture location

10 plunger rod

11 plunger

12 holder

14a particle-free crude plasma

14b crude plasma

15 plunger

16 butterfly cannula

17 mixing vessel

18 substitute and construction agent

19 retaining nose

20 tube

1. A regeneration adjuvant for skin and bone defects, which has:
thrombocyte-rich plasma (platelet-rich-plasma=PRP) and
calcium phosphates, in particular calcium composite, in particular β-tri-calcium phosphate.

2. A regeneration adjuvant as set forth in claim 1 characterized in that the regeneration adjuvant comprises such a mixture of the two components that a pasty shapeable mass is produced.

3. A method of manufacturing a regeneration adjuvant for skin and bone defects comprising the following steps:
a) drawing up blood into a first, in particular elongate container,
b) fixing the first container in a centrifuge (4) with the openable side of the first container directed radially inwardly,
c) carrying out a first centrifuging step,
d) removing the first container from the centrifuge (4), removing the radially inner fraction (crude plasma) from the first container and transferring it into a second, in particular elongate container,
e) fixing the second container in the same centrifuge (4) with the openable side of the second container directed radially inwardly,
f) carrying out a second centrifuging step at a substantially higher and in particular at least double the speed of rotation as in the first centrifuging step,
g) removing the second container from the centrifuge,
h) removing a part of the radially inner fraction (particle-free plasma) from the second container,
i) mixing, in particular by shaking up, the radially outer fraction with the rest of the radially inner fraction in the second tube to give PRP plasma, and
j) mixing that content (PRP plasma) of the second container with a bone substitute or construction agent.

4. A method as set forth in claim 3 characterized in that the first container is a first monovette (1) with piercable sterile closure (3) at one end.

5. A method as set forth in one of the preceding method claims characterized in that the second container is a second monovette (2), onto the sterile closure (3) of which can be fitted an adaptor (5) with a cannula (6), wherein the adaptor (5) has a rearward spike (5a) for piercing or opening the sterile closure (3).

6. A method as set forth in one of the preceding method claims characterized in that the first centrifuging step is effected at least at 900 g and/or the second centrifuging step is effected at at least 1300 g, in particular at at least 1800 g.

7. A method as set forth in one of the preceding method claims characterized in that upon removal of the particle-free plasma so much is removed therefrom that the remaining residue is just sufficient to afford together with the radially outward fraction a viscosity which can be removed by means of a cannula, in particular a liquid.

8. A method as set forth in one of the preceding method claims characterized in that the first and second containers are identical in their external shape, in particular in regard to the fixing regions in the centrifuge (4).

9. A method as set forth in one of the preceding method claims characterized in that the centrifuge (4) has suitable receiving regions for the first container and for the second container.

10. A method as set forth in one of the preceding method claims characterized in that removal of the PRP plasma from the second container is effected by means of a removal tube (7), in particular through the sterile closure (3) of the second monovette (2).

11. A method as set forth in one of the preceding method claims characterized in that the operation of drawing up blood into the first monovette is effected directly from the bloodstream of the patient.

12. A method as set forth in one of the preceding method claims characterized in that after the second centrifuging step upon removal of a part of the radially inner fraction (particle-free plasma) air in a sterile-filtered condition and in particular ambient air is fed into the second container at the same time for volume compensation purposes.

13. A method as set forth in one of the preceding method claims characterized in that upon removal of the PRP plasma from the second container air in a sterile-filtered condition and in particular ambient air is fed into the second container at the same time for volume compensation purposes.

14. A kit for manufacturing a regeneration adjuvant for skin and bone defects from PRP plasma and a bone substitute or construction agent, comprising
a first container, in particular a first monovette (1),
a second container, in particular a second monovette (2),
a centrifuge (4) for holding said containers, in particular monovettes (1, 2), and
a mixing vessel (17) for mixing the PRP plasma obtained with the substitute or construction agent.

15. A kit as set forth in claim 14 characterized in that the kit includes a third container, in particular a tube (20) or a third monovette equipped with a cannula.

16. A kit as set forth in one of the preceding kit claims characterized in that the first monovette (1) has a latching means for its plunger in its rear limit position.

17. A kit as set forth in one of the preceding kit claims characterized in that the first monovette (1) has in its plunger rod (10) a desired-rupture location (9) which when the plunger (11) is completely retracted is arranged outside the container part of the monovette (1), in particular directly outside the container part of the monovette.

18. A kit as set forth in one of the preceding kit claims characterized in that the first container and in particular the first monovette (1) has as the cannula a “butterfly cannula” (16).

19. A kit as set forth in one of the preceding kit claims characterized in that the kit includes at least one and in particular two cannulas (6) equipped with an air sterile filter (13) at the rear end.
